(19) United States
(12) Patent Application Publication
(10) Pub. No.: US 2021/0030379 A1
(43) Pub. Date: Feb. 4, 2021

(54) RADIATION SOURCE AND RADIOGRAPHY APPARATUS

(71) Applicant: FUJIFILM Corporation, Tokyo (JP)

(72) Inventors: Takashi TAJIMA, Ashigarakami-gun (JP); Takeyasu KOBAYASHI, Ashigarakami-gun (JP); Masayoshi MATSUURA, Ashigarakami-gun (JP)

(73) Assignee: FUJIFILM Corporation, Tokyo (JP)

(21) Appl. No.: 16/940,715

(22) Filed: Jul. 28, 2020

(30) Foreign Application Priority Data
Jul. 29, 2019 (JP) 2019-138507

(51) Int. Cl. A61B 6/00

(52) U.S. Cl. CPC A61B 6/4007; A61B 6/4423; A61B 6/4411

(57) ABSTRACT

A radiation source includes: a plurality of radiation tubes that generates radiations; an interval change mechanism that changes an interval between the radiation tubes; and irradiation direction change mechanisms that change irradiation directions in which the radiation tubes emit the radiations.

### CROSS-REFERENCE TO RELATED APPLICATIONS

This application claims priority under 35 U.S.C § 119(a) to Japanese Patent Application No. 2019-138507 filed on 29 Jul. 2019. The above application is hereby expressly incorporated by reference, in its entirety, into the present application.

### BACKGROUND OF THE INVENTION

### 1. Field of the Invention

The present invention relates to a radiation source that generates radiation, such as X-rays, and a radiography apparatus that performs radiography using the radiation source.

### 2. Description of the Related Art

A radiography apparatus that captures an image of an object using radiation, such as X-rays, has come into widespread use. The radiography apparatus comprises, for example, a radiation source that generates radiation and a radiation detection panel that captures an image of an object using the radiation.

Further, the radiography apparatus generally captures the image of a part of the object, such as a specific part of the object. However, some radiography apparatuses can perform so-called long-length imaging. The long-length imaging is imaging in a relatively wide range, such as imaging including a plurality of parts of an object and imaging including substantially the entire spine or lower limb.

In general, a radiography apparatus that performs the long-length imaging performs the long-length imaging by performing imaging once or a plurality of times using a radiation source having one radiation tube. However, a radiography apparatus has been known which performs the long-length imaging using a radiation source having a plurality of radiation tubes (JP2014-057752A and JP2012-066062A (corresponding to US2012/0051513A1)).

### SUMMARY OF THE INVENTION

In a case in which the long-length imaging is performed by one imaging operation using a radiation source having one radiation tube, it is necessary to increase a source-to-image distance (SID). As a result, a large imaging space is required. Therefore, it is difficult to perform the long-length imaging in, for example, a narrow hospital room or a medical examination car. In addition, in a case in which the long-length imaging is performed by a plurality of imaging operations that are performed using a radiation source having one radiation tube while changing an imaging part, the SID can be reduced to the same value as that in normal imaging. However, there is a problem that it is difficult to obtain a sharp image due to the body movement of the object during the plurality of imaging operations. Further, there is a problem that the object is restrained for a long time.

In order to solve these problems, a technique is considered which captures an image of a plurality of parts of an object in a short time, using a radiation source having a plurality of radiation tubes, while reducing the SID to the same value as that in normal imaging.

However, in a case in which the radiation source having a plurality of radiation tubes is used, there is a problem that the size of the radiation source (all of the plurality of radiation tubes) increases.

Further, even in a case in which the radiation source having a plurality of radiation tubes is used, it is difficult to change the arrangement of the plurality of radiation tubes in the related art. As a result, the SID is constant. Therefore, it may be difficult to perform the long-length imaging according to an imaging environment, such as the size of the room where imaging is performed.

Accordingly, an object of the invention is to provide a small radiation source that can flexibly adjust a SID to perform long-length imaging and a radiography apparatus using the radiation source.

According to the invention, there is provided a radiation source comprising: a plurality of radiation tubes that generate radiation; an interval change mechanism that changes an interval between the radiation tubes; and an irradiation direction change mechanism that changes an irradiation direction in which each of the radiation tubes emits the radiation.

Preferably, in a case in which the plurality of radiation tubes are arranged in a first direction, the interval change mechanism changes the interval in the first direction.

Preferably, the interval change mechanism moves the radiation tubes in a second direction perpendicular to the first direction to change an interval between the radiation tube and a radiation detection panel to which the radiation tube emits the radiation.

Preferably, in a case in which the plurality of radiation tubes are arranged in a first direction, some of the plurality of radiation tubes are offset in a second direction perpendicular to the first direction.

Preferably, the radiation source further comprises a fixing member that fixes the radiation source to an imaging room in which radiography is performed.

According to the invention, there is provided a radiography apparatus comprising: the above-mentioned radiation source; a first control unit that controls the interval between the plurality of radiation tubes included in the radiation source and the irradiation direction; a radiography unit including one or more radiation detection panels that capture an image of an object using the radiation; a second control unit that controls radiography using the radiation source and the radiography unit; and an image generation unit that generates a long-length radiographic image using radiographic images obtained from the one or more radiation detection panels.

Preferably, the radiography apparatus further comprises a length measurement unit that measures a length of the object. Preferably, the first control unit changes the interval and/or the irradiation direction using the length of the object.

Preferably, the first control unit increases the interval as the length of the object increases.

Preferably, the first control unit spreads the angle of the irradiation directions as the length of the object increases.

Preferably, the first control unit acquires a source-object distance which is a distance between the radiation source and the object and changes the interval and/or the irradiation direction using the source-object distance.

Preferably, the first control unit increases the interval as the source-object distance increases.

Preferably, the first control unit spreads the angle of the irradiation directions as the source-object distance decreases.

Preferably, the first control unit changes the interval and/or the irradiation direction on the basis of an irradiation field of the radiation source.

Preferably, the first control unit increases the interval as the irradiation field becomes wider.

Preferably, the first control unit spreads the angle of the irradiation directions as the irradiation field becomes wider.

Preferably, the image generation unit corrects the radiographic image obtained from the radiation detection panel according to the interval between the radiation tubes and/or the irradiation direction.

Preferably, the image generation unit corrects the radiographic image obtained from the radiation detection panel on the basis of a correction value that has been recorded in advance.

Preferably, the second control unit controls an order in which the radiation is emitted from the radiation tubes.

Preferably, the second control unit controls the order in which the radiation is emitted for each group including the radiation tubes that are arranged at an interval of one radiation tube or a plurality of radiation tubes.

Preferably, the second control unit performs control such that the radiation is sequentially emitted from first and second groups each including the radiation tubes that are arranged at an interval of one radiation tube.

Preferably, the second control unit resets a portion, in which the radiation emitted from the radiation tubes in the first group and the radiation emitted from the radiation tubes in the second group overlap each other, in the radiation detection panel after the radiation is emitted from the radiation tubes in the first group and before the radiation is emitted from the radiation tubes in the second group.

Preferably, the image generation unit corrects the radiographic image for a radiation overlap portion and generates the long-length radiographic image using the corrected radiographic image.

Preferably, the second control unit controls the order in which the radiation is emitted from the radiation tubes according to a part of the object.

Preferably, the second control unit controls a dose and/or a quality of the radiation emitted from each of the radiation tubes.

Preferably, the second control unit controls the dose and/or the quality of the radiation emitted from each of the radiation tubes according to a part of the object.

Preferably, the radiography apparatus further comprises a radiation dose reduction unit that, in a case in which there is an overlap portion between the irradiation fields of the radiation emitted from the radiation tubes adjacent to each other, reduces the dose of the radiation emitted to the overlap portion.

Preferably, the image generation unit adjusts a density of the radiographic image according to the dose and the quality of the radiation.

Preferably, the radiography apparatus further comprises an irradiation field projection unit that is provided in each of the radiation tubes and projects the irradiation field of the radiation. Preferably, the irradiation field projection unit indicates at least one of the irradiation fields in a different color from other irradiation fields.

The radiation source according to the invention is small and can flexibly adjust the SID. In addition, the radiography apparatus according to the invention can flexibly adjust the SID according to the imaging environment, such as the size of the room where imaging is performed, to perform long-length imaging.

### BRIEF DESCRIPTION OF THE DRAWINGS

FIG. 1 is a diagram schematically illustrating a radiography apparatus.

FIG. 2 is a block diagram illustrating a configuration of a radiation source.

FIG. 3 is a diagram illustrating the arrangement of a plurality of radiation tubes.

FIG. 4 is a diagram illustrating the arrangement of the plurality of radiation tubes and irradiation directions.

FIG. 5 is a diagram illustrating a state in which a SID has been changed.

FIG. 6 is a diagram illustrating a configuration of a long-length imaging radiation source according to the related art.

FIG. 7 is a diagram illustrating arrangement in which some radiation sources are offset in the Z direction.

FIG. 8 is a diagram illustrating arrangement in which some radiation sources are offset in the Y direction.

FIG. 9 is a diagram schematically illustrating a configuration of a radiation source installed in an imaging room.

FIG. 10 is a diagram schematically illustrating a radiography apparatus in a case in which, for example, the SID is automatically controlled.

FIG. 11 is a diagram schematically illustrating a radiography apparatus comprising a length measurement unit.

FIG. 12 is a diagram schematically illustrating a radiography apparatus comprising a distance acquisition unit.

FIG. 13 is a diagram illustrating a distribution of the arrival dose of radiation a radiation detection panel.

FIG. 14 is a diagram illustrating an overlap portion of irradiation fields.

FIG. 15 is a block diagram illustrating a radiation source having an irradiation field projection unit.

### DESCRIPTION OF THE PREFERRED EMBODIMENTS

### First Embodiment

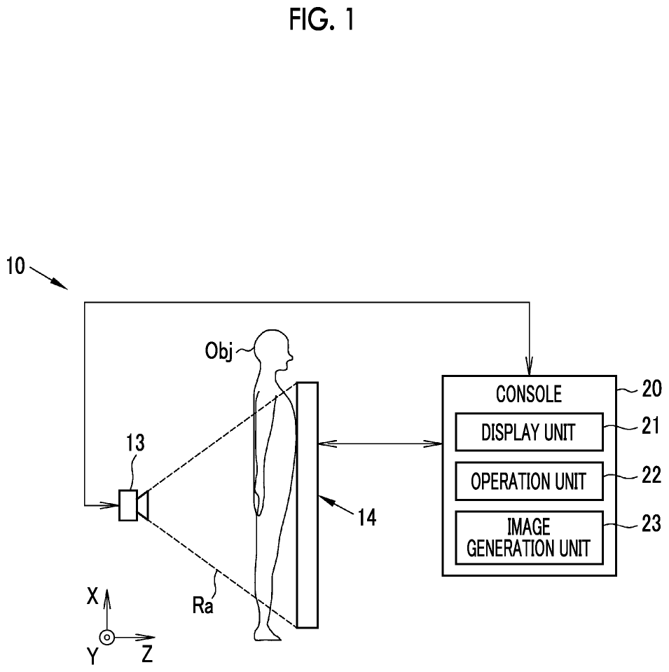

As illustrated in FIG. 1, a radiography apparatus 10 includes a radiation source 13, a radiography unit 14, and a console 20.

The radiography apparatus 10 can perform so-called long-length imaging. The “long-length imaging” is imaging that captures one radiographic image including a long-length object, such as the entire spine or the entire lower limb. Examples of the long-length imaging include imaging that captures one radiographic image using at least two or more radiation detection panels and imaging that separately captures the images of the object two or more times to obtain one radiographic image. In addition, an example of the long-length imaging is imaging that captures one radiographic image including a plurality of parts, such as the head, the chest, the abdomen, the thigh, and the lower leg, as the main objects even in a case in which one radiation detection panel is used. The main object means a part of the object as an imaging target. A part of the object included in, for example, an end portion of the radiographic image is excluded in order to capture the image of the main object. In the following description, it is assumed that radiography performed by the radiography apparatus 10 is the long-length imaging unless otherwise specified. However, the radiography apparatus 10 may perform radiography other than the long-length imaging.

The radiation source 13 is a device that generates radiation Ra required for imaging and consists of, for example, a radiation tube that generates the radiation Ra and a high-voltage generation circuit that generates a high voltage required for the radiation tube to generate the radiation Ra. The radiation source 13 can adjust, for example, a tube voltage and a tube current of the radiation tube to generate a plurality of types of radiations having different qualities (so-called energy distributions). The energy of the radiation generated by the radiation source 13 is one of the imaging conditions. In this embodiment, the radiation source 13 is an X-ray source that generates X-rays. Therefore, the radiography apparatus 10 is an X-ray imaging apparatus that captures an image of an object Obj using X-rays to acquire an X-ray image of the object Obj. The object Obj is, for example, a person.

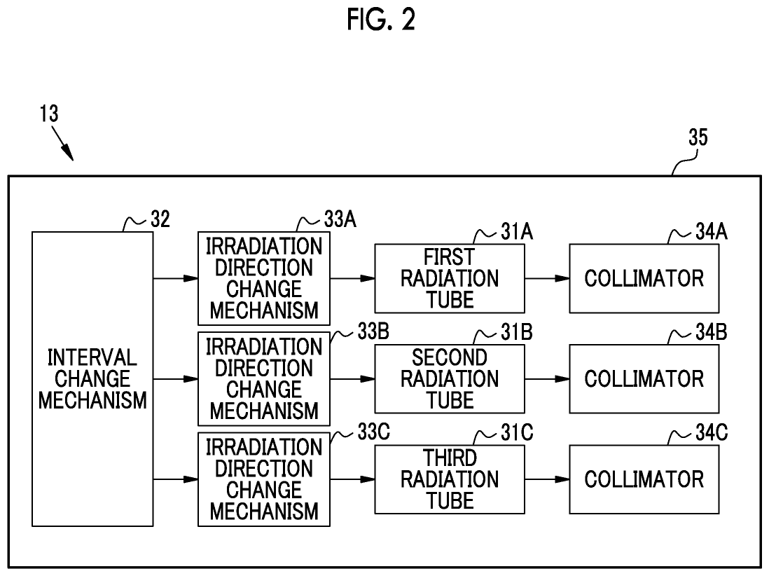

The radiation source 13 comprises a plurality of radiation tubes 31A to 31C (see FIG. 2) that generate radiation. This is for shortening the imaging time and preventing, for example, the blurring of the image captured by the long-length imaging due to the body movement of the object Obj, as compared to a case in which the long-length imaging is performed using one radiation tube while sequentially changing the imaging part. The radiation source 13 uses two or more of the plurality of radiation tubes 31A to 31C at least in the long-length imaging.

In this embodiment, each of the radiation tubes 31A to 31C comprises the high-voltage generation circuit. This is for generating radiation individually from each of the radiation tubes 31A to 31C. However, some or all of the plurality of radiation tubes 31A to 31C forming the radiation source 13 can share the high-voltage generation circuit.

Further, in this embodiment, the plurality of radiation tubes 31A to 31C forming the radiation source 13 comprise collimators 34A to 34C that adjust the irradiation field (irradiation range) of radiation, respectively (see FIG. 2). This is for adjusting the irradiation range of radiation from each of the radiation tubes 31A to 31C. However, the radiation source 13 may have a configuration in which some or all of the radiation tubes 31A to 31C share the collimator.

The radiography unit 14 captures the image of the object Obj using the radiation Ra generated by the radiation source 13. Therefore, the radiography unit 14 includes one or more radiation detection panels that capture the image of the object Obj using the radiation Ra. The radiography unit 14 is a so-called flat panel detector (FPD). Therefore, the radiography unit 14 detects the radiation Ra transmitted through the object Obj and converts the radiation Ra into an electric signal, using the radiation detection panel, and outputs a radiographic image of the object Obj. In imaging using the radiography unit 14, a grid (not illustrated) may be used if necessary. The grid is a device that removes a scattered ray component of radiation and is, for example, a stationary Lysholm blende or a mobile Bucky blende.

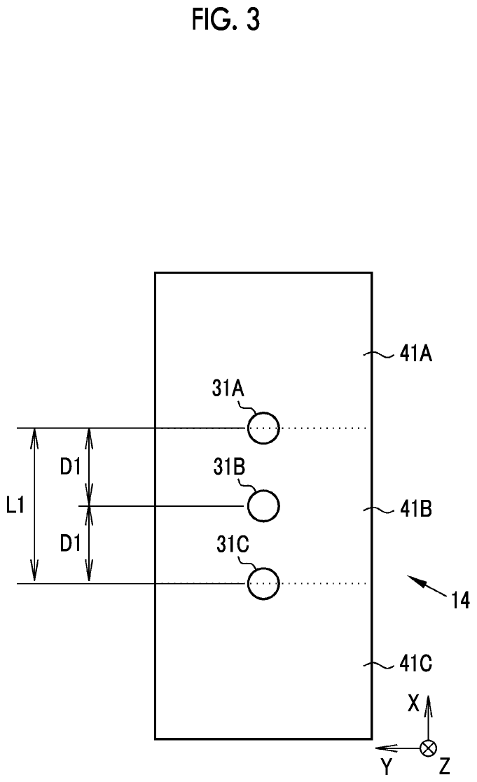

The radiography unit 14 includes one or more radiation detection panels for long-length imaging. In this embodiment, the radiography unit 14 includes a plurality of radiation detection panels 41A to 41C (see FIG. 3). The radiation detection panels 41A to 41C can be individually driven and it is possible to obtain radiographic images from each of the radiation detection panels 41A to 41C. In a case in which the long-length imaging is performed, the radiography apparatus 10 connects and combines the radiographic images acquired from each of the radiation detection panels 41A to 41C to obtain a radiographic image with a long length (hereinafter, referred to as a long-length radiographic image), which is the object of the long-length imaging. The radiography unit 14 can be configured by one large-area radiation detection panel that can accommodate the long-length object Obj.

The radiation detection panels 41A to 41C forming the radiography unit 14 may comprise a plurality of radiation detectors that convert radiation into electric signals if necessary. For example, the radiographic images obtained from each radiation detector are used for a so-called energy subtraction process. Further, the radiation detection panels 41A to 41C forming the radiography unit 14 may be either an indirect conversion type or a direct conversion type. The indirect-conversion-type detector is a detector that indirectly obtains an electric signal by converting the radiation Ra into visible light using a scintillator made of, for example, cesium iodide (CsI) and performing photoelectric conversion for the visible light. The direct-conversion-type detector is a detector that directly converts the radiation Ra into an electric signal using a scintillator made of, for example, amorphous selenium. Any one of a penetration side sampling (PSS)-type detector or an irradiation side sampling (ISS)-type detector can be used in the radiation detection panels 41A to 41C forming the radiography unit 14. The PSS type is a type in which a scintillator is disposed closer to the object Obj than a thin film transistor (TFT) for reading an electric signal. The ISS type is a type in which the scintillator and the TFT are disposed in the order of the TFT and the scintillator from the object Obj, contrary to the PSS type.

The console 20 is a control device (computer) that controls the operation of, for example, the radiation source 13 and the radiography unit 14 and includes, for example, a display unit 21, an operation unit 22, and an image generation unit 23. The display unit 21 is, for example, a liquid crystal display and displays a captured long-length radiographic image, other radiographic images, and necessary information related to other operations or settings. The operation unit 22 is, for example, a keyboard and/or a pointing device that is used to input the settings of imaging conditions and to operate the radiation source 13 and the radiography unit 14. The display unit 21 and the operation unit 22 can be configured by a touch panel.

The image generation unit 23 generates a radiographic image using the output of the radiography unit 14. In a case in which the long-length imaging is performed, the image generation unit 23 generates a long-length radiographic image using the radiographic images obtained from one or more radiation detection panels included in the radiography unit 14. In this embodiment, since the radiography unit 14 has the plurality of radiation detection panels 41A to 41C, radiographic images are generated using the outputs from the radiation detection panels 41A to 41C and the generated radiographic images are connected and combined to generate a long-length radiographic image.

Some or all of the functions of the image generation unit 23 can be provided in an image processing apparatus connected to the console 20. For example, the image processing apparatus can be directly connected to the console 20, can acquire the outputs of the radiation detection panels 41A to 41C in real time, and can be used for the generation of a long-length radiographic image and other radiographic images and image processing. In addition, instead of being directly connected to the console 20, for example, the image processing apparatus may indirectly acquire the outputs of the radiation detection panels 41A to 41C through radiology information systems (RIS), hospital information systems (HIS), picture archiving and communication systems (PACS), or a digital imaging and communications in medicine (DICOM) server included in the PACS and may be used for the generation of a long-length radiographic image and other radiographic images and image processing.

As illustrated in FIG. 2, the radiation source 13 comprises the plurality of radiation tubes 31A to 31C, an interval change mechanism 32, irradiation direction change mechanisms 33A to 33C, and the collimators 34A to 34C. In this embodiment, for simplicity, the radiation source 13 comprises three radiation tubes, that is, the first radiation tube 31A, the second radiation tube 31B, and the third radiation tube 31C. However, the radiation source 13 may comprise two radiation tubes or four or more radiation tubes.

Among the units forming the radiation source 13, at least the radiation tubes 31A to 31C are accommodated in a housing 35. In this embodiment, all of the components including the plurality of radiation tubes 31A to 31C are accommodated in the housing 35. Therefore, the plurality of radiation tubes 31A to 31C are integrated to form one radiation source 13. The size of the radiation source 13 is referred to as the length of the housing 35 in a specific direction.

The interval change mechanism 32 changes the intervals between the radiation tubes 31A to 31C. That is, the radiation source 13 can adjust the intervals between the plurality of radiation tubes 31A to 31C using the interval change mechanism 32. For example, in a case in which the plurality of radiation tubes 31A to 31C are arranged in a first direction, the interval change mechanism 32 changes the intervals in the first direction. In addition, the interval change mechanism 32 moves the radiation tubes 31A to 31C in a second direction perpendicular to the first direction to change the intervals between the radiation tubes 31A to 31C and the radiation detection panels 41A to 41C to which the radiation tubes emit radiation. In this embodiment, the plurality of radiation tubes 31A to 31C are linearly arranged along a specific X direction and the interval change mechanism 32 changes the interval in the X direction.

It is possible to manually or automatically change the intervals between the radiation tubes 31A to 31C using the interval change mechanism 32. Further, the interval change mechanism 32 is configured by a combination of, for example, a rail to which the radiation tubes 31A to 31C are attached, a cam mechanism, a gear, or other mechanical mechanisms. The interval change mechanism 32 can change the intervals between the plurality of radiation tubes 31A to 31C continuously or stepwise. The “intervals” between the plurality of radiation tubes 31A to 31C forming the radiation source 13 are the distances between the plurality of radiation tubes 31A to 31C.

Each of the irradiation direction change mechanisms 33A to 33C changes the irradiation direction in which each of the radiation tubes 31A to 31C emits radiation at any position of each of the radiation tubes 31A to 31C determined by the interval change mechanism 32. That is, the radiation source 13 can adjust the irradiation directions in which the radiation is emitted by the radiation tubes 31A to 31C to any direction using the irradiation direction change mechanisms 33A to 33C. It is possible to manually or automatically change the irradiation directions using the irradiation direction change mechanisms 33A to 33C. The irradiation direction change mechanisms 33A to 33C can change the irradiation directions of the radiation tubes 31A to 31C continuously or stepwise, respectively. The irradiation direction change mechanisms 33A to 33C are configured by a combination of mechanical mechanisms such as gears.

The “irradiation direction” in which the radiation tubes 31A to 31C emit radiation means a direction in which radiation generation intensity is the highest. Therefore, the irradiation direction is determined by the internal structure of the radiation tubes 31A to 31C, such as the inclination direction of an anode and a target, and the arrangement direction of the radiation tubes 31A to 31C in the radiation source 13. Therefore, the irradiation direction change mechanisms 33A to 33C rotate the radiation tubes 31A to 31C to change the irradiation direction of each of the radiation tubes 31A to 31C.

The collimators 34A to 34C are configured using, for example, a plurality of shielding plates (for example, lead plates (not illustrated)) for shielding radiation and the positions of the shielding plates are adjusted to determine the irradiation field of radiation. It is possible to manually or automatically adjust the irradiation field of radiation using the collimators 34A to 34C. In this embodiment, the radiation tubes 31A to 31C comprise the collimator 34A to 34C, respectively. In a case in which the radiation tubes 31A to 31C are moved by the interval change mechanism 32 and are rotated by the irradiation direction change mechanisms 33A to 33C, the collimators 34A to 34C are moved and rotated with the movement of the corresponding radiation tubes 31A to 31C. This is for adjusting the irradiation field of radiation with respect to the irradiation direction in which radiation is emitted from each of the radiation tubes 31A to 31C.

Hereinafter, the operation of the radiation source 13 having the above-mentioned configuration in the long-length imaging will be described. As illustrated in FIG. 3, in this embodiment, the plurality of radiation tubes 31A to 31C included in the radiation source 13 are linearly arranged in the order of the first radiation tube 31A, the second radiation tube 31B, and the third radiation tube 31C from the positive side to the negative side of the X direction along a specific direction (hereinafter, referred to as the X direction, which holds for other figures including, for example, FIG. 1). Further, the direction of a perpendicular line drawn from the radiation source 13 to the radiography unit 14 is referred to as the Z direction and a direction perpendicular to the X direction and the Z direction is referred to as the Y direction (which holds for other figures including, for example, FIG. 1). In this embodiment, the plurality of radiation tubes 31A to 31C are arranged in the XY plane. For example, the plurality of radiation tubes 31A to 31C are moved or rotated in the XY plane by the interval change mechanism 32 and the irradiation direction change mechanisms 33A to 33C.

In this embodiment, the radiography unit 14 comprises three radiation detection panels, that is, the first radiation detection panel 41A, the second radiation detection panel 41B, and the third radiation detection panel 41C. The radiation detection panels 41A to 41C correspond to the radiation tubes 31A to 31C, respectively. That is, the first radiation detection panel 41A captures an image of the object Obj using the radiation emitted by the first radiation tube 31A. The second radiation detection panel 41B captures an image of the object Obj using the radiation emitted by the second radiation tube 31B. Similarly, the third radiography panel 41C captures an image of the object Obj using the radiation emitted by the third radiation tube 31C.

Further, the radiation detection panels 41A to 41C capture the images of different parts of the same object Obj. The reason is that the first radiation detection panel 41A substantially captures an image of a part of the object Obj on the first radiation detection panel 41A, the second radiation detection panel 41B substantially captures an image of a part of the object Obj on the second radiation detection panel 41B, and the third radiation detection panel 41C substantially captures an image of a part of the object Obj on the third radiation detection panel 41C.

The radiation source 13 and the radiography unit 14 can be relatively moved in any direction. However, the radiation source 13, the radiography unit 14, and the object Obj are basically adjusted during imaging. That is, the radiation source 13 faces the radiography unit 14 and the radiation tubes 31A to 31C are arranged substantially at the center of the radiography unit 14 in the X direction and the Y direction.

In this embodiment, the interval change mechanism 32 move the radiation tubes 31A to 31C in the X direction in the radiation source 13 to change the intervals between the plurality of radiation tubes 31A to 31C. In FIG. 3, both the interval between the first radiation tube 31A and the second radiation tube 31B adjacent to each other and the interval between the second radiation tube 31B and the third radiation tube 31C adjacent to each other are “D1”. The length of the arrangement (hereinafter, referred to as an arrangement length) of the plurality of radiation tubes 31A to 31C is “L1”.

A specific arrangement length of the plurality of radiation tubes 31A to 31C during radiography can be changed by the interval change mechanism 32. The maximum value of the arrangement length (hereinafter, referred to as a maximum arrangement length) is determined by the movable range of the plurality of radiation tubes 31A to 31C by the interval change mechanism 32. In addition, the size of the radiation source 13, that is, the size of the housing 35 of the radiation source 13 generally needs to increase as the maximum arrangement length of the plurality of radiation tubes 31A to 31C increases. Therefore, the maximum arrangement length of the plurality of radiation tubes 31A to 31C generally indicates the size of the radiation source 13. Hereinafter, it is assumed that the arrangement length L1 of the radiation tubes 31A to 31C in FIG. 3 is the maximum arrangement length of the plurality of radiation tubes 31A to 31C in the radiation source 13.

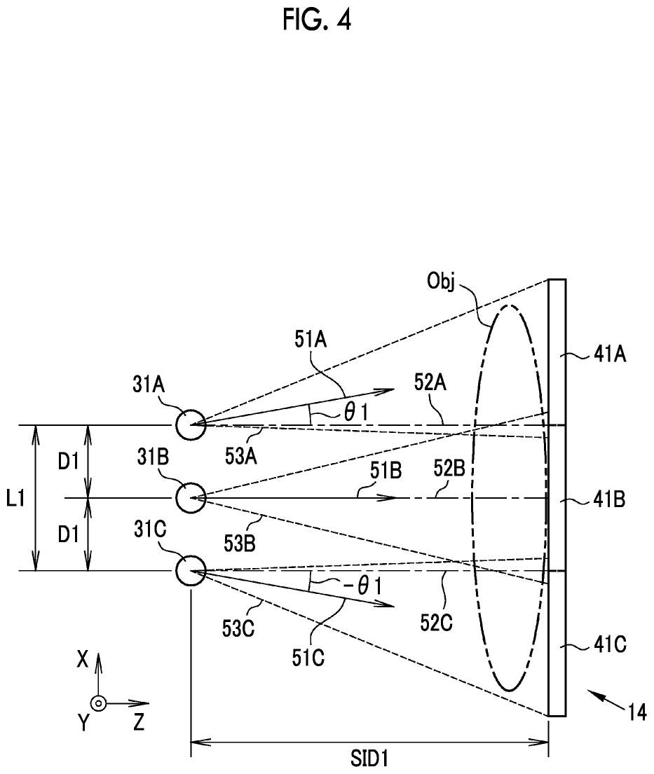

As illustrated in FIG. 4, in a case in which the arrangement length of the radiation tubes 31A to 31C is the maximum arrangement length “L1”, all of the SIDs which are the distances between the plurality of radiation tubes 31A to 31C and the corresponding radiation detection panels 41A to 41C are “SID1” (SID1>0). The irradiation direction change mechanisms 33A to 33C change the irradiation directions by rotating the radiation tubes 31A to 31C about the Y axis at the positions of the radiation tubes 31A to 31C determined by the interval change mechanism 32, respectively, if necessary. In this embodiment, a perpendicular line drawn from each of the radiation tubes 31A to 31C to each of the corresponding radiation detection panel 41A to 41C is used as a reference for rotation in the irradiation direction. The reason is that, in a case in which radiography for obtaining a fluoroscopic image is performed, in general, the irradiation direction of a radiation tube is substantially perpendicular to a corresponding radiation detection panel (a radiation detection panel receiving radiation) in the radiation source according to the related art.

The irradiation direction change mechanism 33A rotates the first radiation tube 31A in the positive direction about the Y axis in the arrangement in which the arrangement length of the radiation tubes 31A to 31C is set to “L1” such that the SID is “SID1”. As a result, the angle of an irradiation direction 51A of the first radiation tube 31A from a perpendicular line 52A drawn from the first radiation tube 31A to the first radiation detection panel 41A is set to “θ1” degrees. Here, “θ1” is a positive number. The first radiation tube 31A whose irradiation direction 51A has been rotated by θ1 degrees emits radiation 53A to the first radiation detection panel 41A during imaging. The irradiation field of the radiation 53A is adjusted by the collimator 34A. Specifically, the irradiation field of the radiation 53A is adjusted according to an effective pixel region of the first radiation detection panel 41A. The effective pixel region is a region including pixels that contribute to a radiographic image. The maximum arrangement length “L1” is less than at least the length of the effective pixel region of the radiography unit 14 (the entire effective pixel regions of the radiation detection panels 41A to 41C).

On the other hand, the irradiation direction change mechanism 33B does not rotate the second radiation tube 31B in the arrangement in which the arrangement length of the radiation tubes 31A to 31C is set to “L1” such that the SID is “SID1”. Therefore, an irradiation direction MB of the second radiation tube 31B is substantially aligned with a perpendicular line 52B drawn from the second radiation tube 31B to the second radiation detection panel 41B. The second radiation tube 31B whose irradiation direction MB has been substantially aligned with the direction of the perpendicular line 52B emits radiation 53B to the second radiation detection panel 41B during imaging. The irradiation field of the radiation 53B is adjusted by the collimator 34B according to the effective pixel region of the second radiation detection panel 41B.

The irradiation direction change mechanism 33C rotates the third radiation tube 31C in the negative direction about the Y axis. As a result, the angle of an irradiation direction MC of the third radiation tube 31C from a perpendicular line 52C drawn from the third radiation tube 31C to the third radiation detection panel 41C is “−θ1” degrees. The third radiation tube 31C whose irradiation direction MC has been rotated by −θ1 degrees emits the radiation 53C to the third radiation detection panel 41C during imaging. The irradiation field of the radiation 53C is adjusted by the collimator 34C according to the effective pixel region of the third radiation detection panel 41C.

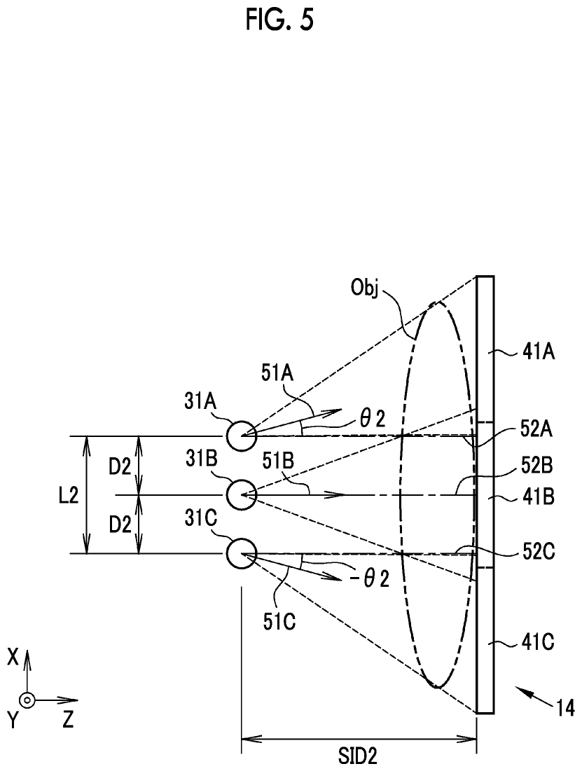

The radiography apparatus 10 can change the SID according to the configuration of the radiation source 13. For example, as illustrated in FIG. 5, imaging can be performed with the SID set to “SID2” shorter than the “SID1” (see FIG. 4). In this case, the interval change mechanism 32 changes the interval between the first radiation tube 31A and the second radiation tube 31B and the interval between the second radiation tube 31B and the third radiation tube 31C to “D2” shorter than “D1” (see FIG. 4) (D1>D2). As a result, the interval change mechanism 32 changes the arrangement length of the plurality of radiation tubes 31A to 31C to “L2” shorter than “L1” (see FIG. 4) (D1>L2).

Then, the irradiation direction change mechanism 33A rotates the first radiation tube 31A about the Y axis to change the angle between the irradiation direction 51A and the perpendicular line 52A to θ2 degrees greater than θ1 degrees (see FIG. 4) (θ1<θ2). The irradiation direction change mechanism 33B maintains the angle of the second radiation tube 31B and maintains the angle between the irradiation direction 51B and the perpendicular line 52B at substantially zero degrees. Further, the irradiation direction change mechanism 33C rotates the third radiation tube 31C about the Y axis to change the angle between the irradiation direction 51C and the perpendicular line 52C to “−θ2” degrees less than “−θ1” degree (see FIG. 4) (−θ1>—θ2).

Regardless of whether the plurality of radiation tubes 31A to 31C simultaneously or sequentially emit the radiations 53A to 53C, all of the radiation 53A emitted from the first radiation tube 31A to the first radiation detection panel 41A, the radiation 53B emitted from the second radiation tube 31B to the second radiation detection panel 41B, and the radiation 53C emitted from the third radiation tube 31C to the third radiation detection panel 41C are the radiation Ra emitted the radiation source 13 to the radiography unit 14.

As described above, the radiation source 13 can change the SID. Then, the SID is changed by changing the intervals between the plurality of radiation tubes 31A to 31C in the radiation source 13 and by changing the irradiation directions 51A to 51C. Therefore, the radiation source 13 can be smaller than the radiation source according to the related art and can flexibly change the SID.

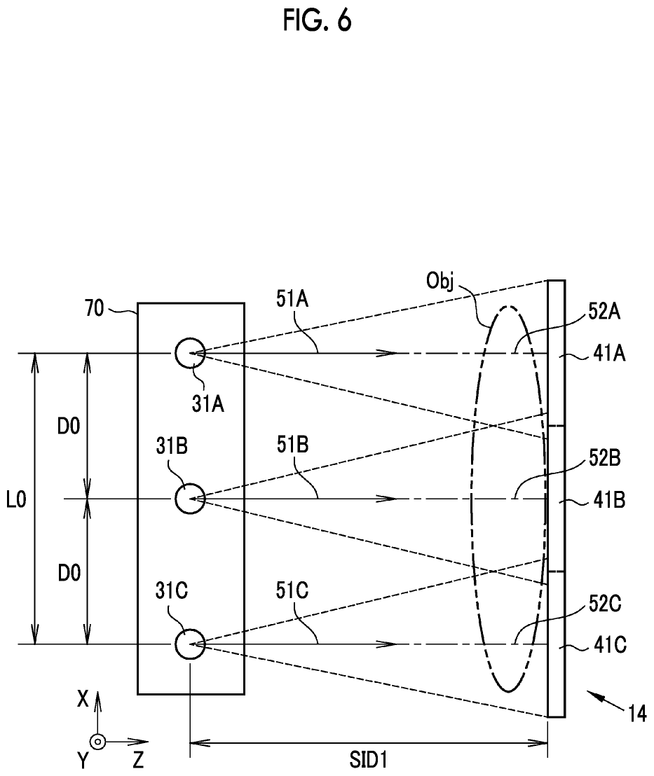

As illustrated in FIG. 6, a long-length imaging radiation source 70 according to the related art includes, for example, a plurality of radiation tubes 31A to 31C. It is difficult to change the intervals between the radiation tubes 31A to 31C and irradiation directions 51A to 51C. Therefore, in the long-length imaging radiation source 70 according to the related art, the radiation tubes 31A to 31C are arranged in front of the corresponding radiation detection panels 41A to 41C, respectively. That is, it is assumed that the irradiation direction 51A of the first radiation tube 31A is the direction of a perpendicular line 52A, the irradiation direction 51B of the second radiation tube 31B is the direction of a perpendicular line 52B, and the irradiation direction 51C of the third radiation tube 31C is the direction of a perpendicular line 52C. Then, for example, in a case in which the SID is set to “SID1” as in FIG. 4, the intervals between the radiation tubes 31A to 31C are “D0” greater than “D1” (see FIG. 4) according to the size of the radiography unit 14 (D1<D0). As a result, in the long-length imaging radiation source 70 according to the related art, the arrangement length of the radiation tubes 31A to 31C is “L0” greater than “L1” (see FIG. 4) (L1<L0).

In contrast, in the radiation source 13, the intervals between the radiation tubes 31A to 31C and the irradiation directions 51A to 51C are variable. Therefore, all of the radiation tubes 31A to 31C do not need to be placed in front of the corresponding radiation detection panels 41A to 41C, respectively. Therefore, in a case in which the same SID is achieved, the arrangement length of the radiation tubes 31A to 31C is shorter than that in the long-length imaging radiation source 70 according to the related art. As a result, the housing 35 of the radiation source 13 can be smaller than that of the long-length imaging radiation source 70 according to the related art.

The SID of the long-length imaging radiation source 70 according to the related art is a substantially fixed value. For example, even in a case in which the collimator is adjusted to simply widen the irradiation field in order to change the SID, it is difficult to obtain a long-length radiographic image used for, for example, diagnosis since a so-called heel effect becomes remarkable. The heel effect (also referred to as a tilt effect) is a phenomenon in which a relative decrease in the dose of radiation or beam hardening occurs on the anode side in the irradiation field of radiation according to the correlation between, for example, the material and shape of an anode forming the radiation tube and the range of use of radiation (the degree of elongation in the irradiation direction) and a shadow that does not depend on the object Obj occurs in a captured radiographic image.

In contrast, in the radiation source 13, it is possible to change the intervals between the plurality of radiation tubes 31A to 31C and the irradiation directions 51A to 51C. In particular, since the irradiation directions 51A to 51C of the radiation tubes 31A to 31C are adjusted, the radiation source 13 can flexibly change the SID while suppressing the heel effect. As a result, according to the radiation source 13 and the radiography apparatus 10 using the radiation source 13, it is easy to obtain a long-length radiographic image that can be used for, for example, diagnosis.

In addition, since the radiation source 13 can flexibly change the SID as described above, it is possible to perform the long-length imaging in a narrow room, such as a hospital room where the object Obj is present or a medical examination car, in addition to imaging performed in a dedicated imaging room where a sufficient imaging space can be secured.

Further, the radiation source 13 and the radiography apparatus 10 using the radiation source 13 can perform the long-length imaging at a shorter SID than the long-length imaging radiation source 70 according to the related art. Therefore, it is possible to reduce the dose of radiation (so-called mAs value) emitted from the radiation tubes 31A to 31C. As a result, since a load on the radiation tubes 31A to 31C of the radiation source 13 is less than that in the long-length imaging radiation source 70 according to the related art, it is possible to increase the lifetime of the radiation tubes 31A to 31C.

### Second Embodiment

In the first embodiment, the plurality of radiation tubes 31A to 31C are arranged along the X direction which is the first direction and the interval change mechanism 32 changes the intervals between the radiation tubes 31A to 31C. However, a method for changing the arrangement and interval of the plurality of radiation tubes 31A to 31C forming the radiation source 13 is not limited thereto.

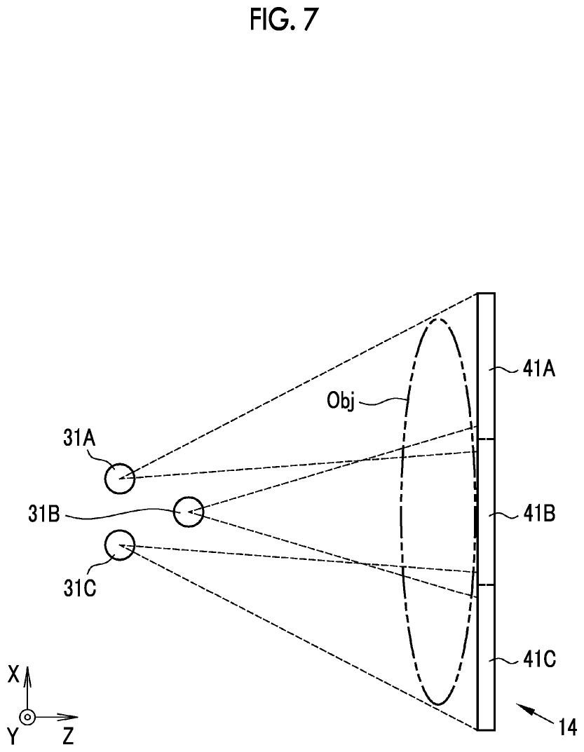

For example, as illustrated in FIG. 7, in a case in which the plurality of radiation tubes 31A to 31C forming the radiation source 13 are arranged along the X direction which is the first direction, some (for example, the second radiation tube 31B) of the plurality of radiation tubes 31A to 31C may be arranged so as to be offset in the Z direction which is the second direction perpendicular to the X direction. In this case, it is possible to reduce mutual physical interference due to the actual sizes of the radiation tubes 31A to 31C. As a result, the plurality of radiation tubes 31A to 31C can be arranged with a shorter arrangement length in the X direction than that in a case in which some radiation tubes are arranged in the XY plane without being offset in the Z direction. Therefore, in a case in which some of the plurality of radiation tubes 31A to 31C are arranged so as to be offset in the Z direction, it is possible to further reduce the size of the radiation source 13. In addition, it is possible to extend the range of the SID that can be adjusted even in a case in which the size is not further reduced.

In FIG. 7, among the plurality of radiation tubes 31A to 31C, the second radiation tube 31B at the center is offset in the Z direction. However, the first radiation tube 31A and the third radiation tube 31C may be offset in the Z direction. Since the plurality of radiation tubes 31A to 31C are relatively offset, the arrangement in which the second radiation tube 31B at the center is offset in the Z direction and the arrangement in which the first radiation tube 31A and the third radiation tube 31C are offset in the Z direction have substantially the same configuration.

In FIG. 7, among the plurality of radiation tubes 31A to 31C, the second radiation tube 31B at the center is offset to the positive side of the Z direction (toward the radiography unit 14). However, the radiation tube that is offset in the Z direction among the plurality of radiation tubes 31A to 31C may be offset to the negative side of the Z direction. Since the plurality of radiation tubes 31A to 31C are relatively offset, the arrangement in which some radiation tubes are offset to the positive side of the Z direction and the arrangement in which some radiation tubes are offset to the negative side of the Z direction have substantially the same configuration. However, as described above, in a case in which the radiation source 13 is configured using the three radiation tubes 31A to 31C, it is preferable that the second radiation tube 31B at the center is relatively offset to the positive side of the Z direction from the first radiation tube 31A and the third radiation tube 31C. The reason is that physical interference is unlikely to occur between the radiation tubes 31A to 31C and the irradiation fields of radiation from the radiation tubes 31A to 31C are unlikely to interfere with each other.

Further, in FIG. 7, the second radiation tube 31B at the center among the plurality of radiation tubes 31A to 31C is offset in the Z direction. However, any radiation tubes that are offset in the Z direction may be selected from the plurality of radiation tubes 31A to 31C. For example, in a case in which the radiation source 13 includes three radiation tubes 31A to 31C, the first radiation tube 31A may be offset with respect to the second radiation tube 31B and the third radiation tube 31C. Similarly, the third radiation tube 31C may be offset in the Z direction with respect to the first radiation tube 31A and the second radiation tube 31B. However, in a case in which the radiation source 13 includes three radiation tubes 31A to 31C, it is preferable that the second radiation tube 31B at the center is relatively offset in the Z direction with respect to the first radiation tube 31A and the third radiation tube 31C. The reason is that physical interference with the first radiation tube 31A and physical interference with the third radiation tube 31C can be removed by the offset of one second radiation tube 31B, which is efficient.

In addition to the above, the interval change mechanism 32 can move the radiation tubes 31A to 31C not only in the X direction which is the first direction but also in the second direction perpendicular to the first direction to change the intervals between the radiation tubes 31A to 31C and the radiation detection panels 41A to 41C to which the radiation tubes 31A to 31C emit radiation. That is, the interval change mechanism 32 can change the distances of the radiation tubes 31A to 31C to the radiation detection panels 41A to 41C. Therefore, even in the configuration in which the plurality of radiation tubes 31A to 31C are arranged in the XY plane as in the first embodiment, in a case in which the intervals are reduced and physical interference occurs between the radiation tubes 31A to 31C, the interval change mechanism 32 may move some of the plurality of radiation tubes 31A to 31C in the Z direction which is the second direction. According to the interval change mechanism 32, it is possible to obtain the arrangement in which some of the radiation tubes are offset in the Z direction if necessary.

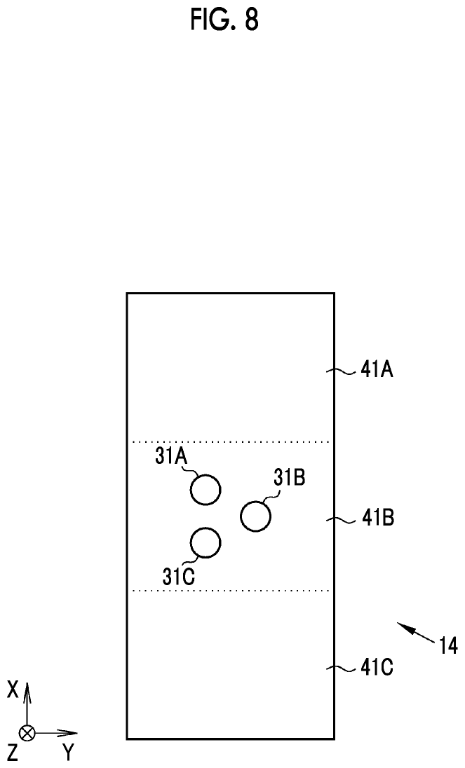

In the second embodiment, the second direction perpendicular to the X direction which is the first direction is the Z direction. However, the second direction may be the Y direction. That is, as illustrated in FIG. 8, some radiation tubes (for example, the second radiation tube 31B) among the plurality of radiation tubes 31A to 31C may be arranged so as to be relatively offset in the Y direction. In this case, it is possible to reduce the physical interference between the radiation tubes 31A to 31C and to further reduce the size of the radiation source 13. Further, the range of the SID that can be adjusted even in a case in which the size is not further reduced is extended. In addition, it is possible to obtain the arrangement in which some radiation tubes are offset in the Y direction by the interval change mechanism 32 if necessary. As described above, the arrangement in which the Y direction is the second direction and some of the plurality of radiation tubes 31A to 31C are offset has an advantage that it is easy to maintain the SIDs of the radiation tubes 31A to 31C in common.

### Third Embodiment

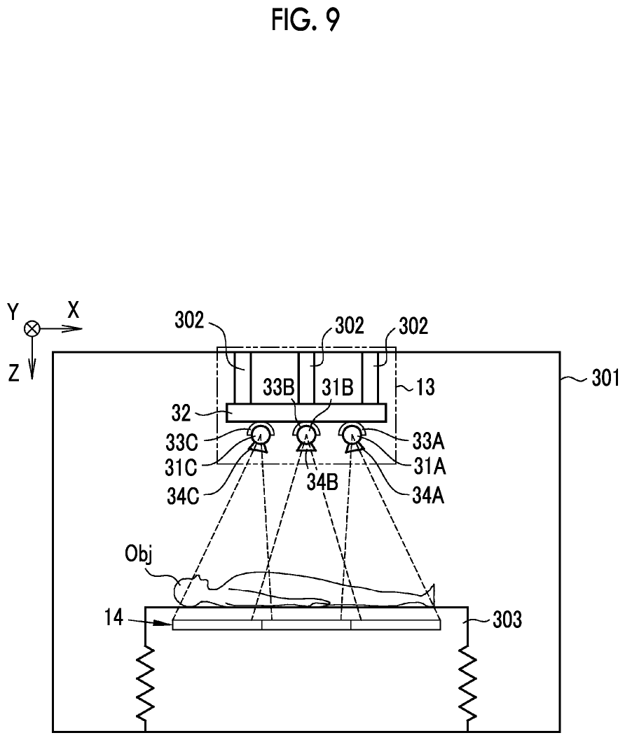

In the first embodiment and the second embodiment, the radiation source 13 is incorporated with the housing 35 and can be moved to any position, for example, in an imaging room only for radiography, a hospital room, or a medical examination car (hereinafter, referred to as an imaging room). However, the radiation source 13 may be installed in an imaging room 301 as illustrated in FIG. 9. In this case, the radiation source 13 comprises fixing members 302 for fixing the radiation source 13 in the imaging room 301 in which radiography is performed, instead of or in addition to the housing 35. The fixing member 302 is, for example, a support or a bolt.

As such, even in a case in which the radiation source 13 is installed in the imaging room 301, the radiation source 13 changes the SID by changing the intervals between the plurality of radiation tubes 31A to 31C included in the radiation source 13 and each of the irradiation directions 51A to 51C. Therefore, the radiation source 13 can be configured to be smaller than the long-length imaging radiation source 70 according to the related art.

In addition, in a case in which the long-length imaging radiation source 70 according to the related art is used, it is difficult to use the function of a bed 303 on which the object Obj is placed even though the height of the bed 303 can be adjusted. However, according to the radiation source 13, even in a case in which the radiation source 13 is installed in the imaging room 301 as described above, it is possible to change the SID. Therefore, it is possible to adjust the height of the bed 303 and to use the bed 303. This is also suitable for a case in which the position of the object Obj with respect to the radiation source 13 is limited in, for example, a hospital room or a medical examination car.

The place where the radiation source 13 is installed in the imaging room 301 is, for example, a ceiling, a floor, or a wall surface of the imaging room 301. In a case in which a long-length image of the object Obj on the bed 303 is captured, it is preferable to install the radiation source 13 on the ceiling or floor of the imaging room 301. In a case in which a long-length image of the object Obj on the bed 303 is captured, it is particularly preferable to install the radiation source 13 on the ceiling. The reason is that the movement or operation of a radiology technician who operates the radiography apparatus 10, the object Obj, or other apparatuses is not hindered. Further, in a case in which an image of the object Obj in the upright position is captured, it is preferable that the radiation source 13 is installed on the wall surface of the imaging room 301.

### Fourth Embodiment

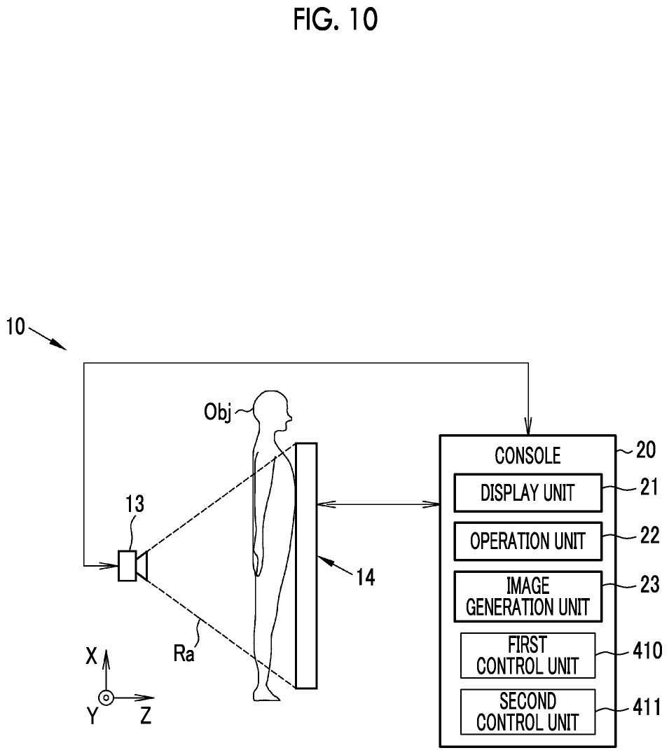

The radiography apparatuses 10 according to the first, second, and third embodiments can automatically perform the operation related to the change of the SID of the radiation source 13. In this case, as illustrated in FIG. 10, the radiography apparatus 10 comprises a first control unit 410 and a second control unit 411 provided in, for example, the console 20.

The first control unit 410 controls the intervals between the plurality of radiation tubes 31A to 31C of the radiation source 13 and the irradiation directions 51A to 51C. As a result, the first control unit 410 automatically adjusts the SID to a SID corresponding to the content of the settings of, for example, an imaging menu. Specifically, the first control unit 410 automatically controls the interval change mechanism 32 and the irradiation direction change mechanisms 33A to 33C of the radiation source 13. Further, the first control unit 410 can control the collimators 34A to 34C of the radiation source 13 to automatically control the irradiation fields of the radiations 53A to 53C emitted from the radiation tubes 31A to 31C. In a case in which the radiography apparatus 10 has a mechanism capable of automatically moving the radiation source 13, the first control unit 410 can move the radiation source 13 to automatically adjust the SID, in addition to the change of the intervals between the radiation tubes 31A to 31C and the irradiation directions 51A to MC in the radiation source 13.

The second control unit 411 controls radiography using the radiation source 13 and the radiography unit 14. The control of the radiography includes, for example, the control of the dose, quality, and emission order of the radiations 53A to 53C emitted from each of the radiation tubes 31A to 31C and the control of the reading, reset, and reading and reset timings of the radiographic images in the radiation detection panels 41A to 41C.

As described above, in the radiography apparatus 10 comprising the first control unit 410 and the second control unit 411, the first control unit 410 changes the intervals between the plurality of radiation tubes 31A to 31C and the irradiation directions 51A to 51C to automatically adjust the SID and the second control unit 411 automatically performs the emission of the radiation Ra and necessary adjustment after the emission.

In the above configuration, the radiation source 13 has the plurality of radiation tubes 31A to 31C and the intervals between the radiation tubes and the irradiation directions can be changed to any value and any direction. However, in some cases, the setting and operation of the radiation source 13 are complicated. Further, in a case in which imaging is performed using the radiation source 13, it may be necessary to adjust the radiations 53A to 53C emitted from the plurality of radiation tubes 31A to 31C, respectively, and to adjust the operation control of the radiation detection panels 41A to 41C receiving the radiations and, for example, the setting and operation of the radiation source 13 may be complicated. Therefore, it is possible to reduce the work load of, for example, the radiology technician by supporting the complicated setting and operation with the first control unit 410 and the second control unit 411 as described above. In addition, it is possible to reduce an error in the setting of, for example, the SID and to accurately perform imaging corresponding to, for example, an imaging menu. In addition, since the time required for imaging can be reduced, it is possible to reduce a load associated with the capture of the image of the object Obj.

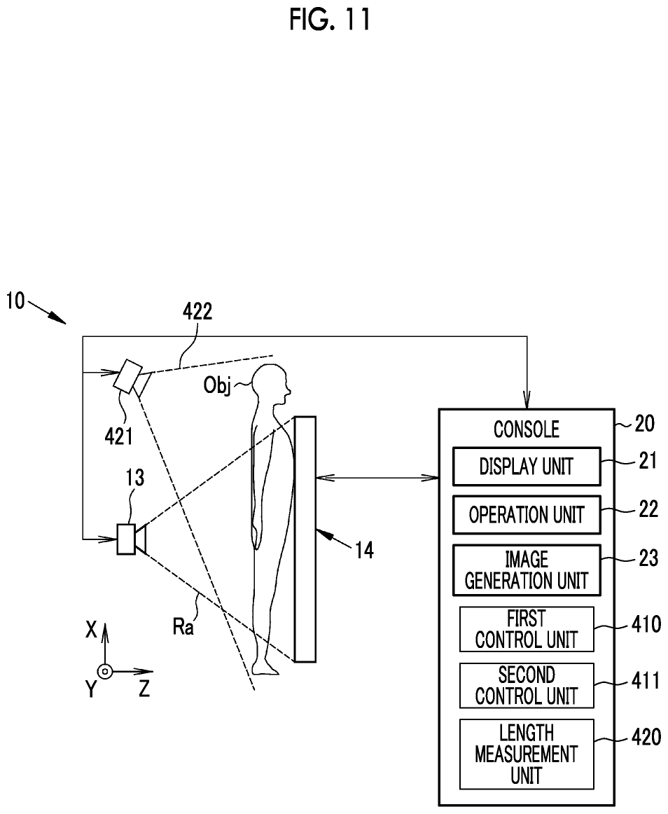

As illustrated in FIG. 11, the radiography apparatus 10 according to the fourth embodiment can comprise a length measurement unit 420 provided in the console 20. The length measurement unit 420 measures the length of the object Obj. Specifically, the length measurement unit 420 obtains an image (hereinafter referred to as a camera image) of the object Obj which has been captured by a camera 421 provided in the imaging room 301 directly or indirectly using visible light, infrared light, or light beams other than radiation. An imaging range 422 of the camera 421 is substantially the entire body of the object Obj, for example, in a state in which radiography can be performed. Therefore, the length measurement unit 420 measures the length of the object Obj using the camera image acquired from the camera 421. The length of the object Obj measured by the length measurement unit 420 is a relative length to, for example, the radiography unit 14 forming the radiography apparatus 10 or the actual size that can be estimated from the length.

In a case in which the length measurement unit 420 is provided as described above, the first control unit 410 changes the intervals between the radiation tubes 31A to 31C and/or the irradiation directions 51A to 51C using the length of the object Obj measured by the length measurement unit 420. Therefore, the radiography apparatus 10 can adjust the SID to a value at which the image of a part of the object Obj can be captured without excess or deficiency, which is the object of the long-length imaging, according to the length of the object Obj.

In a case in which the length measurement unit 420 is provided as described above, the first control unit 410 increases the intervals between the radiation tubes 31A to 31C as the length of the object Obj increases. This is for emitting the radiations 53A to 53C from the radiation tubes 31A to 31C to the front sides of the corresponding radiation detection panels 41A to 41C. In some cases, for example, this configuration makes it possible to reduce the amount of correction required for the radiographic images obtained from each of the radiation detection panels 41A to 41C and the long-length radiographic image generated using the radiographic images.

In a case in which the length measurement unit 420 is provided, the first control unit 410 spreads the angle of the irradiation directions 51A to 51C as the length of the object Obj increases. This is for properly capturing the image of the entire part of the object Obj without excess or deficiency, which is the object of the long-length imaging. The spreading of the angle of the irradiation directions 51A to 51C means increasing the maximum angle formed between the extension lines of the irradiation directions 51A to 51C.

In a case in which the length measurement unit 420 is provided as described above, the first control unit 410 can increase the intervals between the radiation tubes 31A to 31C and spread the angle of the irradiation directions 51A to 51C as the length of the object Obj increases. In addition, the first control unit 410 can increase the intervals between the radiation tubes 31A to 31C according to the length of the object Obj to determine the intervals between the radiation tubes 31A to 31C and can supplementarily adjust the irradiation directions 51A to 51C in order to perform imaging without excess or deficiency. In addition, the first control unit 410 can determine the appropriate irradiation directions 51A to 51C according to the length of the object Obj and then determine the intervals between the radiation tubes 31A to 31C for performing imaging in the determined irradiation directions 51A to 51C without excess or deficiency. In these cases, for example, a radiographic image and a long-length radiographic image that are easy to use for diagnosis are particularly easily obtained.

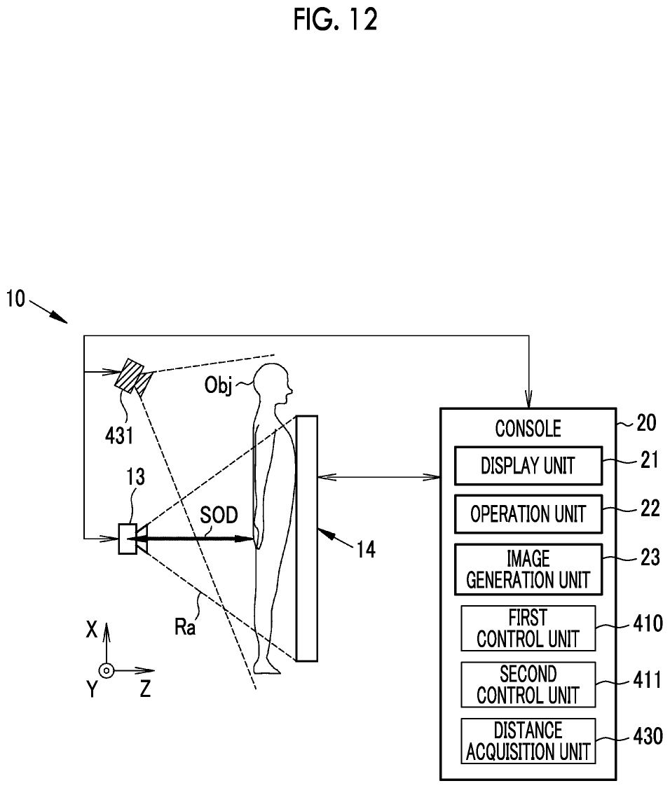

As illustrated in FIG. 12, the radiography apparatus 10 according to the fourth embodiment may comprise a distance acquisition unit 430 provided in the console 20. The distance acquisition unit 430 acquires a source-object distance (so-called SOD) that is a distance between the radiation source 13 and the object Obj. Specifically, the distance acquisition unit 430 directly or indirectly acquires the distance between a distance measurement device 431 provided in, for example, the imaging room 301 and each part of the object Obj from the distance measurement device 431. Then, the source-object distance is obtained using the information of a known positional relationship, such as the distance and directions of the radiation source 13 and the distance measurement device 431. The distance measurement device 431 is, for example, a time-of-flight camera (TOF camera) that measures the time of flight of, for example, infrared rays to measure the distance to an object in a visual field.

In a case in which the distance acquisition unit 430 is provided as described above, the first control unit 410 acquires the source-object distance from the distance acquisition unit 430 and changes the intervals between the radiation tubes 31A to 31C and/or the irradiation directions MA to MC using the source-object distance. Thus, the radiography apparatus 10 can adjust the SID to a SID where the image of a part of the object Obj can be captured without excess or deficiency, which is the object of the long-length imaging, according to the source-object distance.

Specifically, in a case in which the distance acquisition unit 430 is provided, the first control unit 410 increases the intervals between the radiation tubes 31A to 31C as the source-object distance increases. This is for emitting the radiations 53A to 53C from the radiation tubes 31A to 31C to the front sides of the corresponding radiation detection panels 41A to 41C. In some cases, for example, this configuration makes it possible to reduce the amount of correction required for the radiographic images obtained from each of the radiation detection panels 41A to 41C and the long-length radiographic image generated using the radiographic images.

Further, in a case in which the distance acquisition unit 430 is provided, the first control unit 410 spreads the angle of the irradiation directions 51A to 51C as the source-object distance decreases. This is for capturing the entire part of the object Obj without excess or deficiency, which is the object of the long-length imaging.

In a case in which the distance acquisition unit 430 is provided as described above, the first control unit 410 can increase the intervals between the radiation tubes 31A to 31C and spread the angle of the irradiation directions 51A to 51C as the source-object distance increases. In addition, the first control unit 410 can increase the intervals between the radiation tubes 31A to 31C according to the source-object distance to determine the intervals between the radiation tubes 31A to 31C and can supplementarily adjust the irradiation directions 51A to 51C in order to perform imaging without excess or deficiency. In addition, the first control unit 410 can determine the appropriate irradiation directions 51A to 51C according to the source-object distance and can determine the intervals between the radiation tubes 31A to 31C at which imaging is performed in the determined irradiation directions 51A to 51C without excess and deficiency. In these cases, for example, a radiographic image and a long-length radiographic image that are easy to use for diagnosis are particularly easily obtained.

In the above-mentioned modification examples, the distance acquisition unit 430 is provided to acquire the source-object distance. However, in a case in which the first control unit 410 has the function of the distance acquisition unit 430, the first control unit 410 can directly obtain information related to the distance between the distance measurement device 431 and the object Obj from the distance measurement device 431 without passing through the distance acquisition unit 430. That is, the configuration of the distance acquisition unit 430 can be omitted.

Further, it is preferable that the distance measurement device 431 is integrated with the radiation source 13 or is disposed as close to the radiation source 13 as possible. This is for reducing an error of the source-object distance used in the first control unit 410.

In addition, in the radiography apparatus 10 according to the fourth embodiment, the first control unit 410 can change the intervals between the radiation tubes 31A to 31C and/or the irradiation directions 51A to 51C on the basis of the irradiation field of the radiation source 13. The irradiation field of the radiation source 13 is the irradiation range of the radiation Ra (see FIG. 1) and is the entire irradiation field of each of the radiation tubes 31A to 31C. In general, the irradiation field of the radiation source 13 is substantially the effective pixel region of the radiography unit 14. Therefore, in a case in which the size and number (or the size of the bed 303) of radiography units 14 used for imaging or the size and number of radiation detection panels 41A to 41C used for imaging are determined on the basis of, for example, the imaging menu, the irradiation field of the radiation source 13 is also determined. Therefore, the first control unit 410 can acquire information related to the irradiation field of the radiation source 13 on the basis of, for example, the imaging menu and can set an appropriate SID.

As described above, in a case in which the SID is adjusted on the basis of the irradiation field of the radiation source 13, the first control unit 410 increases the intervals between the radiation tubes 31A to 31C as the irradiation field of the radiation source 13 becomes wider. This is for emitting the radiations 53A to 53C from the radiation tubes 31A to 31C to the front sides of the corresponding radiation detection panels 41A to 41C. In some cases, for example, this configuration makes it possible to reduce the amount of correction required for the radiographic images obtained from each of the radiation detection panels 41A to 41C and the long-length radiographic image generated using the radiographic images.

In a case in which the SID is adjusted on the basis of the irradiation field of the radiation source 13 as described above, the first control unit 410 spreads the angle of the irradiation directions 51A to 51C of the radiation tubes 31A to 31C as the irradiation field of the radiation source 13 becomes wider. This is for capturing the entire part of the object Obj without excess or deficiency, which is the object of the long-length imaging.

In a case in which the SID is adjusted on the basis of the irradiation field of the radiation source 13 as described above, the first control unit 410 can increase the intervals between the radiation tubes 31A to 31C and spread the angle of the irradiation directions 51A to 51C as the irradiation field of the radiation source 13 becomes wider. In addition, the first control unit 410 can increase the intervals between the radiation tubes 31A to 31C according to the irradiation field of the radiation source 13 to determine the intervals between the radiation tubes 31A to 31C and then supplementarily adjust the irradiation directions MA to 51C in order to perform imaging without excess or deficiency. Further, the first control unit 410 can determine the appropriate irradiation directions 51A to 51C according to the irradiation field of the radiation source 13 and then determine the intervals between the radiation tubes 31A to 31C for performing imaging in the determined irradiation directions 51A to 51C without excess or deficiency. In these cases, for example, a radiographic image and a long-length radiographic image that are easy to use for diagnosis are particularly easily obtained.

Various modification examples of the fourth embodiment may be combined with each other. That is, the first control unit 410 can change the intervals between the radiation tubes 31A to 31C and/or the irradiation directions 51A to 51C, considering two or more of the length of the object Obj, the source-object distance, and the irradiation field of the radiation source 13.

### Fifth Embodiment

In the radiography apparatus 10 according to the fourth embodiment, it is preferable that the image generation unit 23 corrects the radiographic images obtained from the radiation detection panels 41A to 41C on the basis of the intervals between the plurality of radiation tubes 31A to 31C and/or the irradiation directions 51A to 51C. This is for obtaining a good long-length radiographic image that can be used for, for example, diagnosis.

Specifically, the image generation unit 23 corrects the radiographic images obtained from the radiation detection panels 41A to 41C on the basis of a correction value that has been recorded in advance. The correction value is a target value after correction or a value for, for example, addition, subtraction, multiplication, and division for each pixel or all pixels of the radiographic image in order to obtain a target value after correction. The correction performed by the image generation unit 23 for the radiographic image is, for example, gain correction. The correction value may be acquired or calculated in advance by, for example, calibration or simulation.

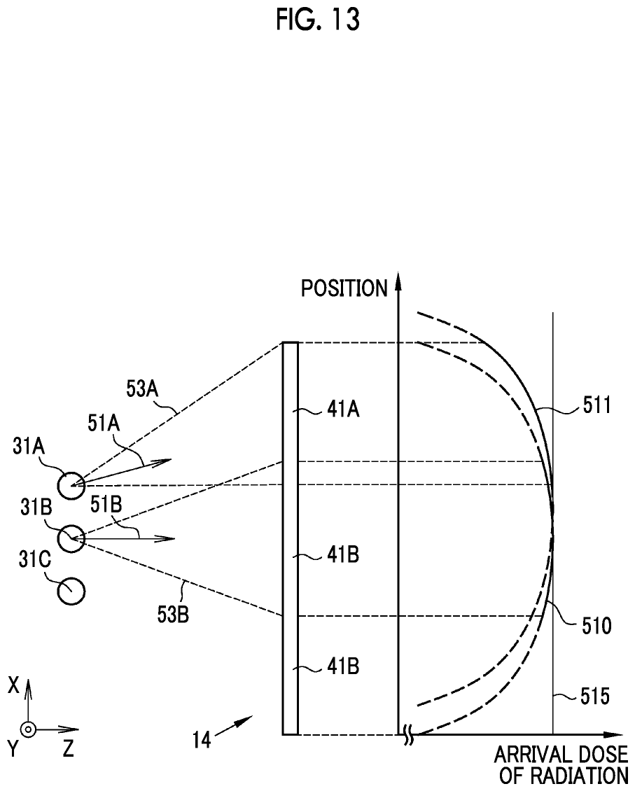

For the radiations 53A to 53C emitted from the radiation tubes 31A to 31C, respectively, as the irradiation field becomes wider, the dose of the radiation reaching the radiation detection panels 41A to 41C (arrival dose) at the end of the irradiation field decreases. In addition, in the radiation source 13, since the positions of the radiation tubes 31A to 31C and the irradiation directions 51A to 51C are different, the distributions of the doses of the radiations 53A to 53C reaching the radiation detection panels 41A to 41C are different from each other. For example, as illustrated in FIG. 13, the distribution of “the arrival dose of the radiation 53A emitted from the first radiation tube 31A” represented by a graph 510 is different from the distribution of “the arrival dose of the radiation 53B emitted from the second radiation tube 31B” represented by a graph 511. This difference is caused by the difference between the position and the irradiation direction 51A of the first radiation tube 31A and the position and the irradiation direction 51B of the second radiation tube 31B. Therefore, it is necessary to correct the radiographic images or the long-length radiographic image in order to obtain a long-length radiographic image captured with the radiation whose arrival dose is the same at any position. The correction is performed in order to obtain a radiographic image and a long-length radiographic image having the same density distribution as that in a case in which imaging is performed with a flat arrival dose regardless of the position, as represented by a graph 515.

Therefore, for example, the image generation unit 23 records the correction value for each of the combinations of the intervals between the plurality of radiation tubes 31A to 31C and the irradiation directions 51A to 51C in advance. Then, the radiographic images from the radiation detection panels 41A to 41C are corrected using an appropriate correction value on the basis of the intervals between the radiation tubes 31A to 31C and the irradiation directions 51A to 51C during imaging to generate a long-length radiographic image. As described above, in a case in which correction correspond to the intervals between the plurality of radiation tubes 31A to 31C and the irradiation directions 51A to 51C is performed to generate a long-length radiographic image, it is possible to obtain a long-length radiographic image suitable for, for example, diagnosis.

The distribution of the arrival dose of radiation in the radiation detection panels 41A to 41C is generated due to not only the generation of radiation as described above but also the heel effect. As described above, in a case in which the correction value is recorded in advance for each of the combinations of the intervals between the plurality of radiation tubes 31A to 31C and the irradiation directions 51A to 51C, it is possible to appropriately correct the distributions including the distribution caused by the heel effect.

Further, in the radiation source 13, there are a considerable number of combinations of the intervals between the plurality of radiation tubes 31A to 31C and the irradiation directions 51A to 51C. Therefore, the correction values for all of these combinations may not be strictly prepared. The image generation unit 23 may calculate correction values for the combinations of the intervals between the radiation tubes 31A to 31C and the irradiation directions 51A to 51C which have not been recorded using, for example, interpolation on the basis of the correction values recorded in association with the intervals between the plurality of radiation tubes 31A to 31C and the irradiation directions 51A to 51C, and may correct the radiographic images using the calculated correction values.

As described above, instead of recording a plurality of correction values in advance for each of the combinations of the intervals between the plurality of radiation tubes 31A to 31C and the irradiation directions 51A to 51C, a correction value corresponding to the longest SID (hereinafter, referred to as a correction value for the longest SID) may be recorded in advance and the radiographic image may be corrected using the correction value. In this case, the accuracy of correction is lower than that in a case in which the correction values for each of the combinations of the intervals between the plurality of radiation tubes 31A to 31C and the irradiation directions 51A to 51C are used. However, according to the correction using the correction value for the longest SID, it is possible to obtain a good radiographic image and a good long-length radiographic image with less incongruity as a whole, regardless of the combinations of the intervals between the plurality of radiation tubes 31A to 31C and the irradiation directions 51A to 51C. Further, since only one correction value for the longest SID longest is sufficient, it is easy to perform, for example, calibration.

### Sixth Embodiment

In the radiography apparatus 10 according to the fourth or fifth embodiment, it is preferable that the second control unit 411 controls the order in which the radiation tubes 31A to 31C emit the radiations 53A to 53C, respectively. The reason is that, in a case in which the second control unit 411 controls the order in which the radiation tubes 31A to 31C emit the radiations 53A to 53C, respectively, it is easy to obtain a good long-length radiographic image that can be used for, for example, diagnosis.

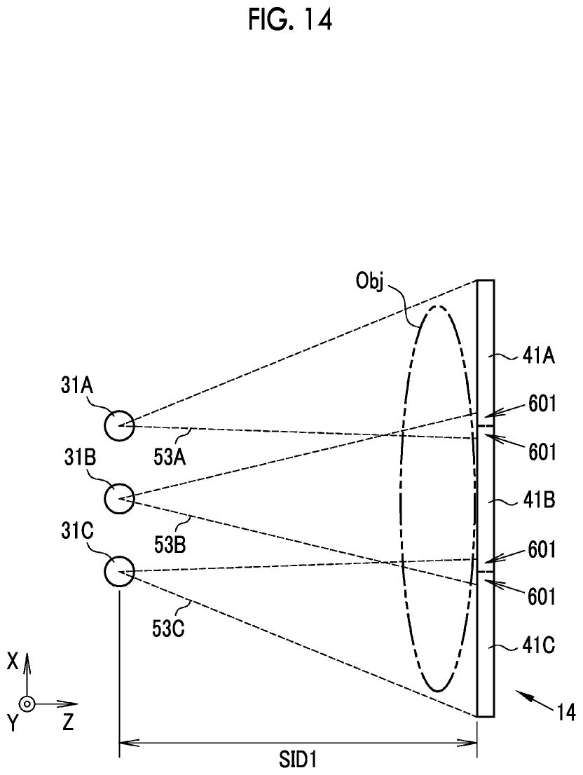

Specifically, the second control unit 411 controls the order in which radiation is emitted for each group including the radiation tubes that are arranged at an interval of one radiation tube or a plurality of radiation tubes. That is, imaging is sequentially performed for each group including the radiation tubes that are not adjacent to each other. In a case in which imaging is performed while radiation is emitted from adjacent radiation tubes at the same time, it is possible to complete the long-length imaging in the shortest time. However, in some cases, radiations emitted from adjacent radiation tubes overlap each other in an adjacent portion or an overlap portion of the effective pixel regions of the radiation detection panels. As illustrated in FIG. 14, in the arrangement in which the SID is set to “SID1”, there is an overlap portion 601 between the irradiation fields of the radiation tubes 31A to 31C. Therefore, in a case in which the radiations 53A to 53C are simultaneously emitted from the radiation tubes 31A to 31C, respectively, for example, the density of the image of the object Obj is disturbed in the overlap portion 601. In contrast, as in this embodiment, in a case in which imaging is sequentially performed for each group including the radiation tubes that are not adjacent to each other, the disturbance of the radiographic image does not occur. As a result, it is easy to obtain a good long-length radiographic image that can be used for, for example, diagnosis. In addition, since imaging is simultaneously performed for each group including the radiation tubes that are not adjacent to each other, it is possible to complete the long-length imaging in a short time without causing the disturbance of the radiographic image.

In a case in which the radiation source 13 has the three radiation tubes 31A to 31C, for example, in the fourth embodiment, it is assumed that the second radiation tube 31B at the center forms a first group and the first radiation tube 31A and the third radiation tube 31C form a second group. Then, radiation is sequentially emitted from each of the first group and the second group including the radiation tubes that are alternately arranged as described above. In particular, it is preferable that the second control unit 411 performs control such that radiation is sequentially emitted from each of the first group and the second group including the radiation tubes that are alternately arranged as described above. In this case, the long-length imaging can be completed by two imaging operations and it is possible to complete imaging in the shortest time in a case in which imaging is sequentially performed using the above-mentioned grouping.

In addition, imaging may be performed using the groups in any order. That is, the second control unit 411 may direct the second radiation tube 31B in the first group to emit the radiation 53B relatively first and obtain a radiographic image using the second radiation detection panel 41B. Then, the second control unit 411 may direct the first radiation tube 31A and the third radiation tube 31C in the second group to simultaneously emit the radiation 53A and the radiation 53C, respectively, and obtain radiographic images from the first radiation detection panel 41A and the third radiation detection panel 41C. In addition, the second control unit 411 may direct the first radiation tube 31A and the third radiation tube 31C in the second group to simultaneously emit the radiation 53A and the radiation 53C, respectively, and obtain radiographic images from the first radiation detection panel 41A and the third radiation detection panel 41C. Then, the second control unit 411 may direct the second radiation tube 31B in the first group to emit the radiation 53B and obtain a radiographic image using the second radiation detection panel 41B. However, in a case in which a part that is likely to cause a defect in a radiographic image due to, for example, the body movement of the object Obj is known in advance, it is preferable that imaging is performed using a group for capturing an image of the part first. That is, the second control unit 411 can control the order in which radiation is emitted from the radiation tubes according to the part of the object Obj. For example, preferably, in a case in which a part in which, for example, body movement is likely to occur is placed at the center of the radiography unit 14, imaging is performed first using the first group. In a case in which the part in which, for example, body movement is likely to occur is placed at one end or both ends of the radiography unit 14, imaging is performed first using the second group. This is for reducing a defect the radiographic image due, for example, to body movement and keeping the influence of the defect within a range in which the defect can be corrected without difficulty.

Further, the second control unit 411 controls the radiation detection panels 41A to 41C. Therefore, in a case in which the radiation tubes are divided into a plurality of groups and imaging is sequentially performed as described above, it is preferable that the second control unit 411 removes (so-called resets) charge at least in the overlap portion 601 of the radiation detection panel corresponding to the radiation tube in the group to be used for imaging later. This is for surely eliminating the influence of the radiation in the previous imaging. For example, in a case in which the radiation tubes are divided into the first group and the second group and imaging is sequentially performed using the two groups, the second control unit 411 resets the overlap portions 601 of the radiation detection panels 41A to 41C, to which radiation is emitted from the radiation tube in the first group and radiation is emitted from the radiation tubes in the second group, after the radiation is emitted from the radiation tube in the first group and before the radiation is emitted from the radiation tubes in the second group.

For example, the amount of overlap between the radiations 53A to 53C in the overlap portions 601 is known from the intervals between the radiation tubes 31A to 31C and the irradiation directions 51A to 51C, and the imaging conditions, such as the dose and quality of each of the radiations 53A to 53C. Therefore, the image generation unit 23 can correct the radiographic images for the overlap portion 601 of the radiations 53A to 53C and generate a long-length radiographic image using the corrected radiographic images. The correction is, for example, correction for changing the density of the overlap portion 601 or correction for reducing one of the overlap images as noise. In a case in which the image generation unit 23 performs the correction, the reset of the overlap portion 601 by the second control unit 411 can be omitted.

### Seventh Embodiment

In the radiography apparatuses 10 according to the fourth, fifth, and sixth embodiments, in addition to the various kinds of control in each of these embodiments, the second control unit 411 can control the dose (specifically, an mAs value) and/or quality (specifically, a tube voltage (kV)) of the radiations 53A to 53C emitted from the radiation tubes 31A to 31C, respectively. In a case in which the second control unit 411 controls the dose and/or quality of the radiations 53A to 53C emitted from the radiation tubes 31A to 31C, respectively, it is easy to obtain a good long-length radiographic image that can be used for, for example, diagnosis.

Specifically, the second control unit 411 can control the dose and/or quality of the radiations 53A to 53C emitted from the radiation tubes 31A to 31C, respectively, according to the part of the object Obj. For example, the dose of radiation emitted from a radiation tube that is used to capture an image of a thin part (for example, the lower leg) of the object Obj is less than the dose of radiation emitted from a radiation tube that is used to capture an image of another thick part (for example, the abdomen) during imaging. As described above, in a case in which the second control unit 411 controls the dose and/or quality of the radiations 53A to 53C emitted from the radiation tubes 31A to 31C, respectively, according to the part of the object Obj, it is possible to avoid unnecessary exposure to the object Obj. In addition, since the image of each part of the object Obj can be captured with an appropriate dose and/or quality, it is possible to obtain a radiographic image and a long-length radiographic image with high contrast for each part of the object Obj.

As described above, in a case in which the dose and/or quality of the radiations 53A to 53C is controlled, the densities of the radiographic images obtained from the radiation detection panels 41A to 41C are different for each part of the object Obj. Therefore, in a case in which the radiographic images captured by partially changing the dose and/or quality of the radiations 53A to 53C are used, the image generation unit 23 adjusts the densities of the acquired radiographic images according to the dose and quality of the radiations 53A to 53C during imaging. Then, a long-length radiographic image is generated using the radiographic images whose densities have been adjusted. This is for obtaining an integrated long-length image without incongruity.

In the radiography apparatus 10 according to each of the above-described embodiments and the modification examples, in a case in which the overlap portion 601 is present between the irradiation fields of the radiations 53A to 53C emitted from the adjacent radiation tubes 31A to 31C (see FIG. 14), it is preferable that the radiography apparatus 10 comprises a radiation dose reduction unit that reduces the dose of radiation emitted to the overlap portion 601. The radiation dose reduction unit is, for example, a portion that is thicker than other portions in an additional member of the collimators 34A to 34C that reduce the dose of radiation reaching the overlap portion 601 or a member (for example, a movable lead plate) that limits the irradiation field in the collimators 34A to 34C.

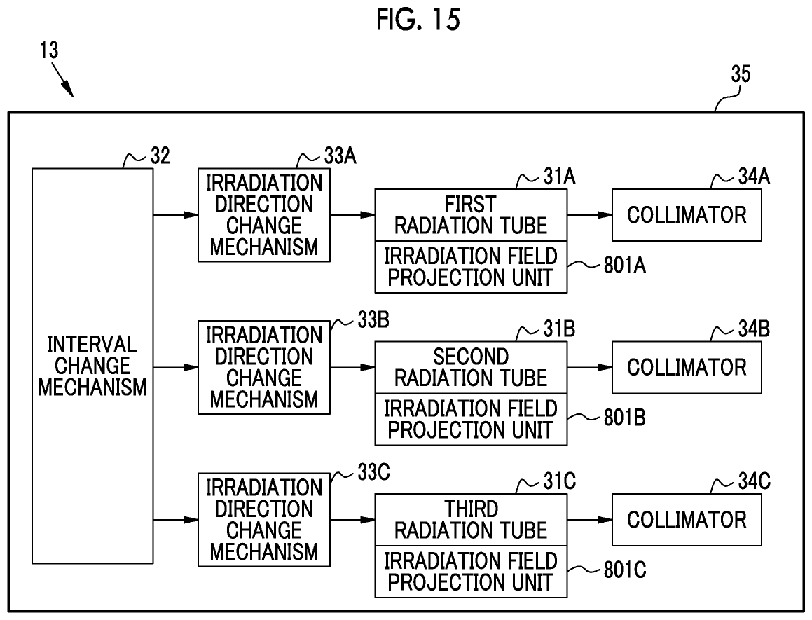

In addition, as illustrated in FIG. 15, it is preferable that the radiography apparatus 10 and the radiation source 13 according to each of the above-described embodiments and modification examples comprise irradiation field projection units 801A to 801C that are provided in the radiation tubes 31A to 31C and project the irradiation fields of the radiations 53A to 53C, respectively. This is for making it easy for, for example, a radiology technician to check the irradiation fields of the plurality of radiation tubes 31A to 31C and the irradiation field of the entire radiation source 13. The irradiation field projection unit 801A projects, for example, the position and size of the irradiation field of the first radiation tube 31A to the radiography unit 14. Similarly, the irradiation field projection unit 801B projects, for example, the position and size of the irradiation field of the second radiation tube 31B to the radiography unit 14 and the irradiation field projection unit 801C projects, for example, the position and size of the irradiation field of the third radiation tube 31C to the radiography unit 14. The irradiation field projection units 801A to 801C are, for example, LEDs or other light emitting elements that project visible light to the radiography unit 14 through the collimators 34A to 34C.

As described above, in a case in which the irradiation field projection units 801A to 801C are provided, it is preferable that the irradiation field projection units 801A to 801C indicate the irradiation field of at least one radiation tube in a different color from the irradiation fields of other radiation tubes. This is for making it easy to visually recognize, for example, the overlap portion 601 between the irradiation fields or the separation of the irradiation fields. This enables, for example, the radiology technician to find or recognize the overlap 601 or the separation of the irradiation fields. Therefore, it is possible to appropriately adjust the irradiation field before the long-length imaging.

In each of the above-described embodiments and modification examples, the following various processors can be used as the hardware structure of processing units performing various processes, such as the image generation unit 23, the first control unit 410, the second control unit 411, the length measurement unit 420, and the distance acquisition unit 430. The various processors include, for example, a central processing unit (CPU) which is a general-purpose processor executing software to function as various processing units, a graphical processing unit (GPU), a programmable logic device (PLD), such as a field programmable gate array (FPGA), which is a processor whose circuit configuration can be changed after manufacture, and a dedicated electric circuit which is a processor having a dedicated circuit configuration designed to perform various processes.

One processing unit may be configured by one of the various processors or a combination of two or more processors of the same type or different types (for example, a combination of a plurality of FPGAs, a combination of a CPU and an FPGA, or a combination of a CPU and a GPU). In addition, a plurality of processing units may be configured by one processor. A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As such, various processing units are configured by using one or more of the various processors as a hardware structure.

In addition, specifically, an electric circuit (circuitry) obtained by combining circuit elements, such as semiconductor elements, can be used as the hardware structure of the various processors. Further, the hardware structure of the storage unit is a storage device such as a hard disc drive (HDD) or a solid state drive (SSD).

### EXPLANATION OF REFERENCES

10: radiography apparatus
13: radiation source
14: radiography unit
20: console
21: display unit
22: operation unit
23: image generation unit
31A: first radiation tube
31B: second radiation tube
31C: third radiation tube
32: interval change mechanism
33A to 33C: irradiation direction change mechanism
34A to 34C: collimator
35: housing
41A: first radiation detection panel
41B: second radiation detection panel
41C: third radiation detection panel
51A to 51C: irradiation direction
52A to 52C: perpendicular line
53A to 53C: radiation
70: long-length imaging radiation source
301: imaging room
302: fixing member
303: bed
410: first control unit
411: second control unit
420: length measurement unit
421: camera
422: imaging range
430: distance acquisition unit
431: distance measurement device
510, 511, 515: graph
601: overlap portion
801A to 801C: irradiation field projection unit

What is claimed is:

1. A radiation source comprising:
a plurality of radiation tubes that generate radiation;
an interval change mechanism that changes an interval between the radiation tubes; and
an irradiation direction change mechanism that changes an irradiation direction in which each of the radiation tubes emits the radiation.

2. The radiation source according to claim 1,
wherein, in a case in which the plurality of radiation tubes are arranged in a first direction, the interval change mechanism changes the interval in the first direction.

3. The radiation source according to claim 2,
wherein the interval change mechanism moves the radiation tubes in a second direction perpendicular to the first direction to change an interval between the radiation tube and a radiation detection panel to which the radiation tube emits the radiation.

4. The radiation source according to claim 1,
wherein, in a case in which the plurality of radiation tubes are arranged in a first direction, some of the plurality of radiation tubes are offset in a second direction perpendicular to the first direction.

5. The radiation source according to claim 1, further comprising:
a fixing member that fixes the radiation source to an imaging room in which radiography is performed.

6. A radiography apparatus comprising:
the radiation source according to claim 1;
a first control unit that controls the interval between the plurality of radiation tubes included in the radiation source and the irradiation direction;
a radiography unit including one or more radiation detection panels that capture an image of an object using the radiation;
a second control unit that controls radiography using the radiation source and the radiography unit; and
an image generation unit that generates a long-length radiographic image using radiographic images obtained from the one or more radiation detection panels.

7. The radiography apparatus according to claim 6, further comprising:
a length measurement unit that measures a length of the object,
wherein the first control unit changes the interval and/or the irradiation direction using the length of the object.

8. The radiography apparatus according to claim 7,
wherein the first control unit increases the interval as the length of the object increases.

9. The radiography apparatus according to claim 7,
wherein the first control unit spreads the angle of the irradiation directions as the length of the object increases.

10. The radiography apparatus according to claim 6,
wherein the first control unit acquires a source-object distance which is a distance between the radiation source and the object and changes the interval and/or the irradiation direction using the source-object distance.

11. The radiography apparatus according to claim 10,
wherein the first control unit increases the interval as the source-object distance increases.

12. The radiography apparatus according to claim 10,
wherein the first control unit spreads the angle of the irradiation directions as the source-object distance decreases.

13. The radiography apparatus according to claim 6,
wherein the first control unit changes the interval and/or the irradiation direction on the basis of an irradiation field of the radiation source.

14. The radiography apparatus according to claim 13,
wherein the first control unit increases the interval as the irradiation field becomes wider.

15. The radiography apparatus according to claim 13,
wherein the first control unit spreads the angle of the irradiation directions as the irradiation field becomes wider.

16. The radiography apparatus according to claim 6,
wherein the image generation unit corrects the radiographic image obtained from the radiation detection panel according to the interval between the radiation tubes and/or the irradiation direction.

17. The radiography apparatus according to claim 16,
wherein the image generation unit corrects the radiographic image obtained from the radiation detection panel on the basis of a correction value that has been recorded in advance.

18. The radiography apparatus according to claim 6,
wherein the second control unit controls an order in which the radiation is emitted from the radiation tubes.

19. The radiography apparatus according to claim 18,
wherein the second control unit controls the order in which the radiation is emitted for each group including the radiation tubes that are arranged at an interval of one radiation tube or a plurality of radiation tubes.

20. The radiography apparatus according to claim 19,
wherein the second control unit performs control such that the radiation is sequentially emitted from first and second groups each including the radiation tubes that are arranged at an interval of one radiation tube.

21. The radiography apparatus according to claim 20,
wherein the second control unit resets a portion, in which the radiation emitted from the radiation tubes in the first group and the radiation emitted from the radiation tubes in the second group overlap each other, in the radiation detection panel after the radiation is emitted from the radiation tubes in the first group and before the radiation is emitted from the radiation tubes in the second group.

22. The radiography apparatus according to claim 18,
wherein the image generation unit corrects the radiographic image for a radiation overlap portion and generates the long-length radiographic image using the corrected radiographic image.

23. The radiography apparatus according to claim 18,
wherein the second control unit controls the order in which the radiation is emitted from the radiation tubes according to a part of the object.

24. The radiography apparatus according to claim 6,
wherein the second control unit controls a dose and/or a quality of the radiation emitted from each of the radiation tubes.

25. The radiography apparatus according to claim 24,
wherein the second control unit controls the dose and/or the quality of the radiation emitted from each of the radiation tubes according to a part of the object.

26. The radiography apparatus according to claim 6, further comprising:
a radiation dose reduction unit that, in a case in which there is an overlap portion between the irradiation fields of the radiation emitted from the radiation tubes adjacent to each other, reduces the dose of the radiation emitted to the overlap portion.

27. The radiography apparatus according to claim 24,
wherein the image generation unit adjusts a density of the radiographic image according to the dose and the quality of the radiation.

28. The radiography apparatus according to claim 6, further comprising:
an irradiation field projection unit that is provided in each of the radiation tubes and projects the irradiation field of the radiation,
wherein the irradiation field projection unit indicates at least one of the irradiation fields in a different color from other irradiation fields.
